(19) United States
(12) Patent Application Publication
(10) Pub. No.: US 2025/0064370 A1
(43) Pub. Date: Feb. 27, 2025

(54) PASS-THROUGH ELECTRODES, DEVICES AND SYSTEMS INCLUDING PASSTHROUGH ELECTRODES, AND METHODS FOR MAKING AND USING SUCH DEVICES

(71) Applicants: Stephen A. Leeflang, Sandy, UT (US); Steven Mickelsen, Palo Alto, CA (US); Blake Allan, Palo Alto, CA (US)

(72) Inventors: Stephen A. Leeflang, Sandy, UT (US); Steven Mickelsen, Palo Alto, CA (US); Blake Allan, Palo Alto, CA (US)

(21) Appl. No.: 18/817,154

(22) Filed: Aug. 27, 2024

Related U.S. Application Data
(63) Continuation of application No. PCT/US2024/043782, filed on Aug. 25, 2024.
(60) Provisional application No. 63/534,580, filed on Aug. 25, 2023.
(60) Provisional application No. 63/633,834, filed on Apr. 14, 2024.
(60) Provisional application No. 63/647,623, filed on May 15, 2024.

(51) Int. Cl. A61B 5/287; A61B 18/14; A61M 25/00

(52) U.S. Cl. CPC A61B 5/287; A61B 18/1492; A61B 2562/0209; A61B 2562/125; A61M 25/0082

(57) ABSTRACT

A pass-through electrode device includes a tubular member including a proximal end, a distal end sized for introduction into a patient's body, a lumen extending between the proximal end and the distal end, and one or more pass-through electrodes on the distal end. Each pass-through electrode includes an outer portion on an outer wall of the tubular member, an inner portion exposed within an inner wall of the lumen, and is passive, e.g., electrically isolated and/or unconnected to other electrodes or components of the device. A system is also provided that includes an outer tubular member including one or more pass-through electrodes and a procedure device introducible through and deployable from the tubular member for performing a procedure, e.g., mapping and/or ablation. The procedure device includes one or more active electrodes that may be aligned with pass-through electrodes when the device is at least partially positioned within the outer member.

### RELATED APPLICATION DATA

The present application is a continuation of co-pending International Application No. PCT/US2024/043782, filed Aug. 25, 2024, which claims benefit of co-pending U.S. provisional application Ser. No. 63/534,580, filed Aug. 25, 2023, 63/633,834, filed Apr. 14, 2024, and 63/647,623, filed May 15, 2024, the entire disclosures of which are expressly incorporated by reference herein.

### TECHNICAL FIELD

The present application relates generally to medical devices and, more particularly, to pass-through electrodes for sheaths, guide catheters, or other tubular devices, e.g., to enhance functionality of mapping and ablation systems, and to methods for making and using such pass-through electrodes, tubular devices, and systems.

### BACKGROUND

Anatomical mapping is ubiquitous in the cardiac electrophysiology (EP) suite. The use of magnetic and impedance-based position sensing elements with or without electrical mapping has transformed EP work, improving outcomes and workflows. The value they provide is so large that even the most innovative new products must incorporate them or risk loss of market share. Their use hasn't just transformed traditional ablation and mapping catheters, but their use in traditionally simple systems such as deflectable sheaths and introducers has been the subject of focus and many product attempts for over a decade.

In the specific case of introducer sheaths (fixed curve or deflectable sheaths), the landscape is littered with failed sheath products that have incorporated position sensors, primarily impedance-based electrodes. The reasons for the failures are easily summed up; they are expensive both in components (e.g., connectors, pigtails, more complex packaging, etc.) but even more so due to poor yields, both the propensity to break during use as well as fail during manufacturing-all of which incur large costs that must ultimately be factored into their use.

The root cause of the poor yields is also quite simple; incorporation of conductors from the electrodes back to the handle/connector through the thin wall of the shaft of the sheath is challenging and, even in basic use of introducer systems, the stresses (bending, torquing, compression, etc.) cause path length changes unequally between the sheath and the conductors, thus causing fractures in the conductors and/or the joint of the conductors to the electrodes. Such incorporation is much easier in classic EP ablation or mapping catheters where the main lumen is used for the passage of the conductors (instead of as a passageway to deliver devices) thus shielding them from substantive path length changes during bending.

Furthermore, the electrical connectors and required cables are also expensive. For example, the connector is typically the single most expensive element on a sheath or guide catheter. Continued attempts to develop and launch sheaths and guide catheters with mapping capabilities are an ongoing testament to the value of this functionality.

Ablation and mapping catheters used widely throughout EP procedures incorporate magnetic and impedance-based position sensors over a significant portion of the distal end of the device. This provides the ability to see the position and/or bend(s) in the catheter proximal to the typical therapeutic elements more distally. This helps to gauge true position, contact angle and/or force, and general intuition of what is going on to the operating physician. When fully extended beyond the distal tip the introducer system (e.g., deflectable sheath, fixed curve sheath or otherwise), sensing electrodes function as intended. However, when retracted into the introducer system, the sensing electrodes do nothing.

Further, in cases where the EP catheter is fully extended, the need for further visualization from the introducer system provides little or no value. However, in many common instances, the preferred delivery of the therapeutic or mapping system requires the EP catheter to be retracted substantially into the sheath, again, rendering the impedance based elements proximal to the distal termination of the introducer system useless.

It is therefore clear that devices, systems, and methods that can provide mapping functionalities without the cost or performance issues of the current state of the art would be useful.

### SUMMARY

The present application is directed to medical devices and, and, more particularly, to pass-through electrodes for sheaths, guide catheters, or other tubular devices, e.g., to enhance functionality of mapping and ablation systems, and to methods for making and using such pass-through electrodes, tubular devices, and systems.

To solve the aforementioned problems and take advantage of clinical situations described above, the present application discloses designs for and methods for using “pass-through” and/or “mapping” electrodes, which may be included on sheaths or other tubular devices for introduction into a patient's body. Pass-through electrodes are generally “passive” electrodes, for example, have no conductors and/or otherwise are electrically isolated from other electrodes and/or components of the devices on which they are provided. In one example, the simplest function of pass-through electrodes on a sheath or other tubular member is to permit passage of electrical signals to impedance-based “active” electrodes positioned within the tubular member from the greater blood pool in a manner substantially the same as if the tubular member surrounding the active electrodes were not present. To say it differently, from an electrical standpoint, inclusion of pass-through electrodes on a sheath or other tubular member of an introducer system and their method of use (e.g., substantial alignment of the active electrodes within the pass-through electrodes) makes the introducer system electrically transparent to the active electrodes positioned inside the sheath.

Further, in one example, passive (pass-through) electrodes may be used in conjunction with a dilator device or system, e.g., by providing one or more active (mapping) electrodes on a dilator that may be covered by an introducer sheath including one or more pass-through electrodes. Such mapping electrodes on the dilator, when covered by an introducer sheath, would otherwise be useless but for the surrounding pass-through electrodes. Further, including active electrodes coupled to conductors extending through the body of the dilator may be substantially simpler than in incorporating such electrodes into conventional introducer systems, e.g., introducers, sheaths or deflectable sheaths.

In another example, the sheaths or tubular members including pass-through electrodes may also include one or more active electrodes. Thus, the sheath may include both passive and active electrodes that may be used separately or in conjunction with one another during a procedure. In yet another example, a tubular member including a plurality of pass-through electrodes may include dedicated passive electrodes and one or more passive electrodes that may be coupled to a conductor to also allow the electrode(s) to function as active electrodes, if desired. For example, a device including mapping electrodes also include an electrode that may function as a “pass-through” electrode example above except now the electrode is connected to a conductor to allow the electrode to also function as an active mapping electrode. The conductors may be positioned triaxially along a braided shaft with a connection at the handle, e.g., as disclosed in U.S. Pat. Nos. 9,427,551, 10,071,222, 10,124,145, and 11,305,092, the entire disclosures of which are expressly incorporated by reference herein.

Various constructions and configurations of pass-through electrodes may be provided on sheaths or other tubular members. For example, the pass-through electrodes may be constructed as a ring or other annular shape, a spot or point electrode, and/or as a combination of the two. Alternatively the sheath, introducer, or guide may be constructed of in part or substantially of conductive material to provide electrical transparency. Generally, each pass-through electrode includes an outer portion on an outer wall of the tubular member and an inner portion on an inner wall of the tubular member, e.g., exposed within an instrument lumen of the tubular member. In one example, the inner portion may be a point or spot electrode, and the outer portion may be a ring electrode or annular member extending around an outer wall of the tubular member.

Pass-through electrodes configured as spot and ring electrodes have different strengths and weaknesses, e.g., depending on the purpose of a procedure catheter or other device introduced through a sheath or other tubular member incorporating pass-through electrodes. In generally a point or spot electrode for the inner portion may minimize disruption of the low friction liners/surfaces/coatings provided on instrument lumens, e.g., to facilitate introduction of procedure catheters. For the outer portion, a ring electrode or annular surface may provide greater symmetry and/or more uniform distribution of electrical energy delivered through a pass-through electrode from an active electrode aligned with or proximate to the pass-through electrode. Alternatively, point or spot electrodes may be provided for the outer portion, e.g., circumferentially and/or axially offset from one another along a length of the sheath or tubular member, which may provide additional orientation/position feedback regarding the procedure device positioned within the tubular member.

In accordance with one example, an apparatus is provided for performing a procedure within a patient's body that includes a tubular member comprising a proximal end, a distal end sized for introduction into a patient's body, a longitudinal axis extending between the proximal end and the distal end; a primary lumen extending between the proximal end and the distal end; and one or more pass-through electrodes on the distal end, each pass-through electrode including an outer portion exposed on an outer wall of the tubular member and an inner portion exposed within an inner wall of the primary lumen.

In accordance with another example, an apparatus is provided for performing a procedure within a patient's body that includes a tubular member comprising a proximal end, a distal end sized for introduction into a patient's body, a longitudinal axis extending between the proximal end and the distal end; a primary lumen extending between the proximal end and the distal end; and a plurality of more pass-through electrodes spaced apart from one another on the distal end, each pass-through electrode including an outer portion exposed on an outer wall of the tubular member and an inner portion exposed within an inner wall of the primary lumen, thereby providing a conductive path between the outer and inner portions.

Optionally, a first pass-through electrode may be coupled to conductor extending proximally from the distal end such that, when the conductor is isolated, the sole conductive path is the conductive path between the outer and inner portions, and when electricity is delivered through the conductor to the at least one pass-through electrode, the at least one pass-through electrode is configured to operate as an active electrode. Thus, when the conductor is inactive, the sole conductive path of the first pass-through electrode is between the outer portion and the inner portion. Thus, the first pass-through electrode may be a combination of a passive and an active electrode depending on whether the conductor is open or closed to a source of electrical energy. In one example, the conductor may be a dedicated conductor extending from the first pass-through electrode to a connector on the proximal end, e.g., such that the first pass-through conductor may be coupled to a controller similar to other active electrodes. Alternatively, the conductor may be a wire or other element that has another function but may also be used as a conductor for the first pass-through conductor, such as a pull or steering wire, a reinforcement wire, and the like.

In accordance with still another example, a dilator is provided for accessing a body lumen of a patient, e.g., through septum or other tissue barrier that includes a tubular member comprising a proximal end, a distal end sized for introduction into a patient's body and terminating in a tapered distal tip for advancing the distal end through an opening in tissue, a lumen extending between the proximal and distal ends; and one or more active electrodes on the distal end configured to perform a diagnostic or therapeutic procedure. In one example, a plurality of electrodes may be spaced apart from one another on the distal end and the distal end may be configured to be introduced into a sheath or other tubular member that includes one or more pass-through electrodes that may be aligned with or positioned proximate to the electrodes on the dilator distal end.

In accordance with another example, a system is provided for performing a procedure within a patient's body that includes a tubular member comprising a proximal end, a distal end sized for introduction into a patient's body to a target location, a lumen extending between the proximal end and the distal end, and one or more pass-through electrodes on the distal end, each pass-through electrode including an outer portion exposed on an outer wall of the tubular member and an inner portion exposed within an inner wall of the primary lumen; and a procedure device comprising a distal portion introducible through the lumen to deploy one or more active electrodes on the distal portion within the target location to perform a diagnostic or therapeutic procedure, the one or more electrodes configured to be aligned with the one or more pass-through electrodes when the distal portion is positioned at least partially within the distal end of the tubular member.

In accordance with yet another example, a system is provided for performing a procedure within a patient's body that includes a tubular member comprising a proximal end, a distal end sized for introduction into a patient's body to a target location, a lumen extending between the proximal end and the distal end, and an electrically conductive region on the distal end; and a procedure device comprising a distal portion introducible through the lumen to deploy one or more active electrodes on the distal portion within the target location to perform a diagnostic or therapeutic procedure, the one or more electrodes configured to be aligned with the conductive region when the distal portion is positioned at least partially within the distal end of the tubular member to allow mapping or sensing using the active electrodes through the conductive region.

In accordance with another example, a method is provided for making a pass-through electrode on a tubular member that includes providing a tubular body including a wall surrounding a lumen; positioning a rivet-like member including a conductive head and a shaft extending from the head within the lumen; inserting the shaft through an opening extending through the wall until the shaft is exposed at an outer surface of the wall and the head is positioned against an inner surface of the wall; and forming an outer portion on the outer surface that is electrically coupled to the head.

In accordance with still another example, a method is provided for making a pass-through electrode on a tubular member that includes providing first and second sections of a tubular body including a wall surrounding a lumen; providing a ring electrode comprising a central region and collars extending from opposite ends of the central region; and attaching the collars to the first and second sections to provide the central region between the first and second sections.

In accordance with yet another example, a method is provided for making a pass-through electrode on a tubular member that includes providing a tubular body including a wall surrounding a lumen; inserting a wire through an opening extending through the wall such that a first end of the wire is exposed at an outer surface of the wall and a second end of the wire is exposed at an inner surface of the wall within the lumen; forming an outer portion of a pass-through electrode on the outer surface using the first end of the wire; and forming an inner portion of the pass-through electrode on the inner surface using the second end of the wire.

In accordance with another example, a method is provided for performing a medical procedure that includes introducing a distal end of a tubular member into a body lumen within a patient's body, the distal end including one or more pass-through electrodes providing a conductive pathway between an outer surface of the distal end and an inner surface of a lumen within the tubular member; introducing a distal end of a procedure device into the lumen such that one or more active electrodes on the procedure device distal end are positioned within the tubular member distal end; positioning the procedure device such that the one or more active electrodes are aligned with or proximate to respective one or more pass-through electrodes on the tubular member; and delivering electrical signals to the one or more active electrodes, the pass-through electrodes transmitting the signals to tissue surrounding the tubular member distal end.

In accordance with yet another example, a method is provided for performing a medical procedure that includes introducing a distal end of a tubular member into a body lumen within a patient's body, the distal end including a plurality of pass-through electrodes providing a conductive pathway between an outer surface of the distal end and an inner surface of a lumen within the tubular member, the pass-through electrodes offset axially and/or circumferentially relative to one another; introducing a distal end of a procedure device into the lumen such that one or more active electrodes on the procedure device distal end are positioned within the tubular member distal end; and positioning the procedure device such that the one or more active electrodes are aligned with or proximate to respective pass-through electrodes on the tubular member to determine a position of the procedure device within the patient's body.

In accordance with still another example, a method is provided for performing a medical procedure that includes introducing a distal end of a tubular member into a body lumen within a patient's body, the distal end including a plurality of pass-through electrodes providing a conductive pathway between an outer surface of the distal end and an inner surface of a lumen within the tubular member, the pass-through electrodes offset axially and/or circumferentially relative to one another; introducing a distal end of a dilator into the lumen such that one or more active electrodes on the procedure device distal end are positioned within the tubular member distal end and a distal tip of the dilator extends from the tubular member distal end; and manipulating the dilator and tubular member together to insert the distal tip of the dilator through an opening in tissue, while transmitting signals to the one or more active sensors and receiving signals from the one or more active sensors to provide position information regarding the dilator within the patient's body, the one or more pass-through electrodes transmitting the signals between the one or more active electrodes and tissue adjacent the tubular member distal end.

Other aspects and features of the present invention will become apparent from consideration of the following description taken in conjunction with the accompanying drawings.

### BRIEF DESCRIPTION OF THE DRAWINGS

It is believed the present invention will be better understood from the following description of certain examples taken in conjunction with the accompanying drawings, in which like reference numerals identify the same elements and in which:

FIG. 1A shows an example of a system for performing a medical procedure including a procedure catheter including a plurality of electrodes, and an outer sheath including one or more pass-through electrodes.

FIG. 1B is a longitudinal cross-section of the system of FIG. 1A taken along 1B-1B with the procedure catheter positioned within the sheath to align electrodes on the catheter with pass-through electrodes on the sheath.

FIG. 2A is a detail of a distal portion of an introducer sheath including a plurality of pass-through ring electrodes spaced apart axially from one another.

FIG. 2B is a cross-section of the introducer sheath of FIG. 2B taken across plane 2B-2B.

FIG. 2C is a longitudinal cross-section of the sheath of FIG. 2A showing construction of pass-through electrodes mounted on the sheath.

FIG. 2D is a longitudinal cross-section of the sheath of FIG. 2A showing an alternative construction for the pass-through electrodes.

FIG. 3A is a detail of a distal portion of another introducer sheath including a plurality of spot or point electrodes spaced apart axially from one another and axially aligned with one another.

FIG. 3B is a cross-section of the introducer sheath of FIG. 3A taken across plane 3B-3B.

FIG. 3C is a longitudinal cross-section of the introducer sheath of FIG. 3A showing an alternative construction of spot or point electrodes.

FIG. 4A is a detail of a distal portion of yet another introducer sheath including a plurality of spot or point electrodes spaced apart axially from one another and offset circumferentially with one another.

FIG. 4B is a cross-section of the introducer sheath of FIG. 4A taken across plane 4B-4B.

FIG. 5 is a detail of a distal portion of still another introducer sheath including a plurality of spot or point electrodes spaced apart axially from one another.

FIGS. 6A-6D show an exemplary method for mounting a pass-through spot electrode to a tubular body.

FIG. 7A shows another example of a system including a deflectable sheath including pass-through electrodes and a dilator introducible through the sheath that includes a plurality of active electrodes.

FIG. 7B is a longitudinal cross-section of the system of FIG. 7A with the dilator positioned within the sheath to align electrodes on the catheter with pass-through electrodes on the sheath.

FIG. 8 is a perspective view of an exemplary single-piece electrode that may be used for a pass-through electrode.

FIGS. 9A-9C show alternative methods for mounting the electrode of FIG. 8 to a sheath or other tubular member.

FIG. 10 shows another exemplary single-piece electrode that may be used for a pass-through electrode.

FIGS. 11A and 11B are cross-sectional views of a sheath or tubular member include multiple single-piece electrodes similar to the electrodes shown in FIG. 10 attached to the tubular member.

FIGS. 12A and 12B are perspective and side views of an example of a ring electrode that may be used for a pass-through electrode.

FIG. 13 shows a distal end of a sheath including a ring electrode, such as the electrode shown in FIGS. 12A and 12B, mounted between sections of the sheath.

FIG. 14 is a cross-sectional view of another example of a ring electrode attached between sections of a sheath.

FIG. 15 is a cross-sectional view of another example of a ring electrode attached between sections of a sheath.

The drawings are not intended to be limiting in any way, and it is contemplated that various examples of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

Before the examples are described, it is to be understood that the invention is not limited to particular examples described, as such may, of course, vary. It is also to be understood that the terminology used herein is for the purpose of describing particular examples only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials are now described.

It must be noted that as used herein and in the appended claims, the singular forms “a,” “an,” and “the” include plural referents unless the context clearly dictates otherwise. Thus, for example, reference to “a compound” includes a plurality of such compounds and reference to “the polymer” includes reference to one or more polymers and equivalents thereof known to those skilled in the art, and so forth.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

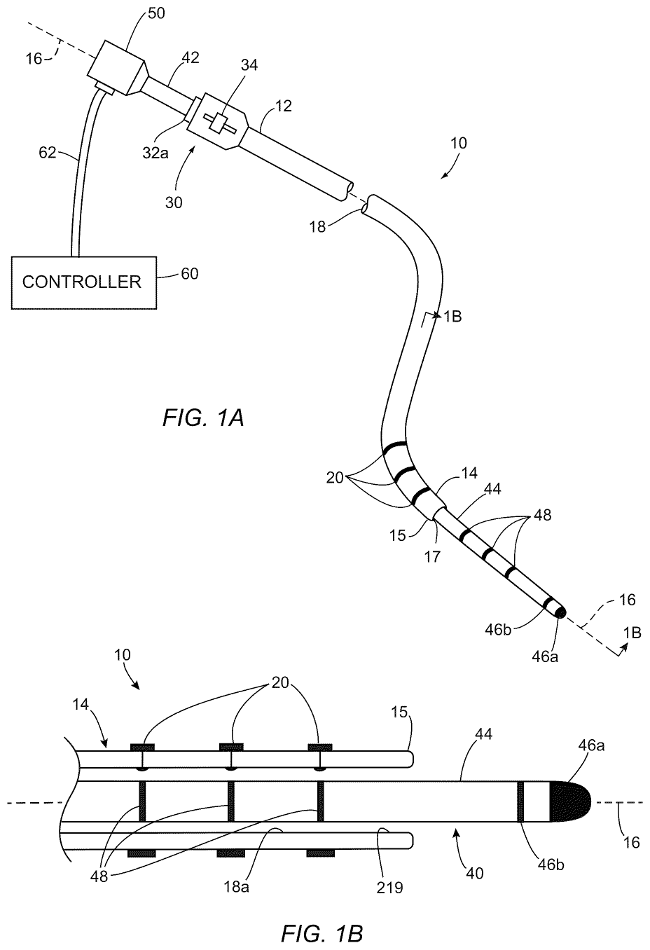

Turning to the drawings, FIGS. 1A and 1B show an example of an apparatus or system 8 for performing a medical procedure, e.g., a mapping and/or ablation procedure, within a patient's body. Generally, as shown in FIG. 1A, the system 8 includes an outer sheath, introducer, guide, or other tubular member 10 and a procedure catheter or device 40 introducible through the sheath 10 to perform the procedure, as described further elsewhere herein.

Generally, the sheath 10 includes a proximal end 12, a distal end 14 sized for insertion into a body lumen, and defining a longitudinal axis 16 extending between the proximal and distal ends 12, 14. In addition, the sheath 10 includes one or more lumens 18 extending at least partially between the proximal and distal ends 12, 14. For example, with additional reference to FIG. 1B, the sheath 10 may include a central or primary lumen 18a extending from the proximal end 12 to an outlet 17 in the distal end 14. The primary lumen 18a may be sized for receiving the procedure catheter 40 and/or other devices. Optionally, the sheath 10 may include one or more additional lumens (not shown), e.g., for receiving one or more steering elements, wires or other electrical conductors, and the like (also not shown).

In addition, as shown in FIGS. 1A and 1B, the sheath 10 includes one or more “pass-through” electrodes 20, i.e., electrodes that include an outer surface or portion 22 exposed on an outer surface 11 of the sheath 10, and an inner surface or portion 24 exposed within the primary lumen 18a that are electrically coupled to one another, e.g., by a wire or other conductor 26 extending through the wall of the sheath 10. In the example shown, the sheath 10 includes three pass-through electrodes 20 spaced apart axially from one another, although the sheath may include fewer (one or two) or more (four or more) pass-through electrodes. As described further elsewhere herein, the pass-through electrodes 18 may be ring electrodes, spot electrodes, or a combination thereof, or have other geometric configurations, e.g., depending on the intended application for the system 8.

The pass-through electrodes 20 are generally “passive,” i.e., they are electrically isolated from one another and from other components of the sheath 10, e.g., such that the sole conductive path of each pass-through electrode is between the outer portion 22 and the inner portion 24. For example, although the outer and inner portions 22, 24 are electrically coupled by the conductor 26, the conductor 26 may be electrically insulated to prevent contact with wires or braid elements (not shown) in the wall of the sheath 10 to enhance isolation of the pass-through electrode 20. Thus, the pass-through electrodes 20 are not coupled to any wires or conductors within the sheath 10, e.g., unlike “active” electrodes that may be provided on the procedure catheter 40, as described further elsewhere herein.

Generally, the pass-through electrodes 20 may be formed from highly conductive materials, such as gold, tungsten, and platinum. However, given that the pathway between the active electrodes on the procedure device inside the sheath and the blood pool is so short and the resistance of blood relatively large compared to typical conductive materials, alternatively, the electrodes may be made from a wide variety of less-conductive metals, such as stainless steel, and/or conductive polymers. Optionally, the pass-through electrodes may include coatings to reduce impedance at the interface with blood or tissue, e.g., as is known in the art.

Returning to FIG. 1B, the distal end 14 may include a tapered, rounded, or otherwise shaped distal tip 15, e.g., to provide a substantially atraumatic tip and/or to facilitate advancement or navigation through various anatomy. Optionally, the distal end 14 may include one or more therapeutic and/or diagnostic elements, e.g., one or more active electrodes, sensors, and the like (not shown), depending upon the particular intended application for the system 8. Further, in addition or alternatively, the distal end 14 may include one or more markers or other features to enhance radiopacity and/or visibility under ultrasound, MRI, or other imaging modalities, e.g., by mounting one or more platinum elements on the distal end 14, doping one or more regions of the distal end 14 with tungsten or barium sulfate, and/or other known methods.

With continued reference to FIG. 1A, the sheath 10 may include a handle or hub 30 on the proximal end 12, e.g., configured and/or sized for holding and/or manipulating the sheath 10 from the proximal end 12. In addition, the handle 30 may include one or more ports, e.g., a port 32a communicating with the primary lumen 18a, or other respective lumens (not shown). Optionally, the port 32a may include one or more valves, e.g., a hemostatic valve (also not shown), which may provide a substantially fluid-tight seal, while accommodating insertion of the procedure catheter 40 or one or more other instruments or fluids into the primary lumen 18a. Optionally, as shown in FIG. 1A, a side port 32b may be provided on the handle 30, e.g., for delivering fluid into and/or aspirating fluid from the primary lumen 18a, e.g., around the catheter 40 or other instrument inserted into the primary lumen 18a through the first port 32a. Optionally, the side port 32b may include one or more connectors, such as a luer lock connector (not shown) for connecting other devices to the side port 32b, such as a syringe or other source of fluid (also not shown).

In addition, the handle 30 may include one or more actuators, such as sliders, buttons, switches, rotational actuators, and the like, e.g., for activating and/or manipulating components (also not shown) on the distal end 14 or otherwise operating the sheath 10. For example, as shown in FIG. 1A, if the distal end 14 is deflectable, an actuator 34 may be provided that is coupled to a proximal end of a steering element within a steering lumen (not shown) extending within the wall of the sheath 10 to the distal end 14. The actuator 34 may be movable, e.g., slidable axially, rotated around the axis 16, and the like, to apply axial, e.g., proximal, tension to the steering element, e.g., to cause the distal end 14 to deflect, as described elsewhere herein.

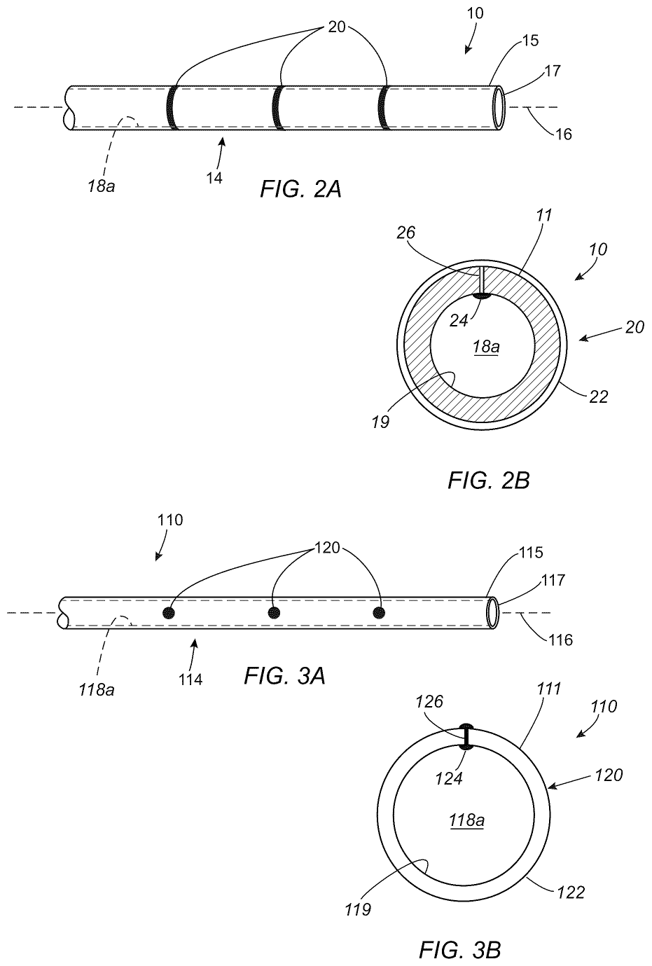

Generally, the sheath 10 is a tubular body constructed to include an inner liner, e.g., at least partially or entirely surrounding or otherwise defining the primary lumen 18a, a reinforcement layer surrounding the inner liner, and an outer jacket surrounding and/or encasing the reinforcement layer (not shown, see, e.g., FIGS. 2C, 2D, and 3C for examples of such multiple-layer construction), each of which may extend at least partially between the proximal and distal ends 12, 14 of the sheath 10. In one example, the inner liner may be formed from lubricious material, e.g., PTFE or a fluoropolymer, to provide a lubricious inner surface for the primary lumen 18a. Alternatively, the inner liner 40 may be formed from one or more layers of thermoplastic or other polymeric material including one or more coatings on the inner surface having desired properties, e.g., a hydrophilic and/or lubricious coating, e.g., similar to the liners disclosed in U.S. Pat. Nos. 7,550,053 and 7,553,387, and U.S. Publication No. 2009/0126862, the disclosures of which are expressly incorporated by reference herein.

One or more of the layers of the sheath 10 may have a substantially homogenous construction between the proximal and distal ends 12, 14. For example, the reinforcement layer may be applied substantially continuously between the proximal and distal ends 12-14. Alternatively, the construction may vary along the length of the sheath 10 to provide desired properties, e.g., between proximal, intermediate, and distal portions (not shown). For example, a proximal portion of the sheath 10 adjacent the proximal end 12 may be substantially rigid or semi-rigid, e.g., providing sufficient column strength to allow the distal end 14 of the sheath 10 to be pushed or otherwise manipulated from the proximal end 12, while a distal portion, e.g., carrying the pass-through electrodes 18,4 may be substantially flexible to accommodate bending and/or introduction into tortuous anatomy.

Returning to FIG. 1A, the procedure catheter 40 may be an elongate member including a proximal end 42 and a distal end 44 sized for introduction into and through the primary lumen 18a of the sheath 10. The catheter 40 may include one or more “active” electrodes 46, 48 on the distal end 40 for performing a medical procedure within a patient's body, e.g., a mapping and/or ablation procedure within a patient's heart (not shown), as described elsewhere herein. For example, as shown, the catheter 40 includes a tip electrode 46a on the distal tip of the catheter 40 and a plurality of ring electrodes 46b, 48 spaced apart from one another proximal to the tip electrode 46a.

As used herein, “active” electrodes, unlike “passive” pass-through electrodes, are electrically coupled to one or more wires or other conductors, which are coupled, in turn, to a controller for delivering signals or energy to the electrodes to perform a diagnostic and/or therapeutic procedure. For example, the electrodes 46, 48 shown in FIG. 1A may be coupled to wires (not shown) extending proximally from the distal end 44 of the catheter 40 to the proximal end 42, where the wires may be coupled to a controller 60. The controller 60 may be coupled to the electrodes 46, 48 for delivering and receiving signals to map conductive pathways within a heart chamber within which the distal end 34 is introduced and/or delivering electrical energy to tissue to ablate the tissue. As shown, the catheter 40 may include a handle or hub 50 on the proximal end 42, which may include one or more connectors (not shown) for connecting to a cable 62 to electrically couple the electrodes 46, 48 to the controller 60.

The controller 60 may be configured to deliver and receive signals from the electrodes 46, 48 and process the signals to identify conductive pathways in adjacent tissue and/or otherwise identify desired anatomical structures. As described further elsewhere herein, the pass-through electrodes 20 are arranged on the distal end 14 of the sheath 10 such that, when the active ring electrodes 48 are positioned within the distal end 14 and aligned with or proximate to respective pass-through electrodes 20, e.g., as shown in FIG. 1B, the pass-through electrodes 20 may be electrically coupled to the respective active electrodes 48 to deliver and receive the signals to/from the controller 60 to perform mapping and/or ablation even without deploying the active electrodes 48 distally from the distal end 14 of the sheath 10.

In one example, the inner portions 24 of the pass-through electrodes 20 may extend from the inner wall 19 of the lumen 18a, e.g., to define a dome or other convex shape or may be concave, e.g., conforming to the curvature of the inner wall 19. The distal end 44 of the catheter 40 and the lumen 18a may have respective diameters that are sufficiently close that the inner portions 24 slidably contact the outer surface of the catheter 40. Thus, when the pass-through electrodes 20 are axially aligned with the active electrodes 48, there may be a light interference fit between the electrodes 20, 48, e.g., to provide direct electrical contact may be made to transmit and receive signals and/or center or otherwise stabilize the catheter 40 within the lumen 18a. Alternatively, the relative diameters may be such that at least some of the inner portions 24 are spaced away from the respective active electrodes 48 when they are axially aligned. In this alternative, blood or other fluid, e.g., saline, within the primary lumen 18a around the catheter 40 may provide sufficient conductive pathways between the pass-through electrodes 20 and closest active electrodes 48.

In either alternative, signals received by the controller 60 may be used to identify when the active electrodes 48 are sufficiently axially aligned with respective pass-through electrodes 20. For example, if the active electrodes 48 are offset axially from the pass-through electrodes 20, the signals received by the controller 60 may not correspond to expected signals when the active electrodes 48 receive signals from adjacent tissue e.g., if the distal end 44 of the catheter 40 were deployed from the sheath 10. Once the electrodes 20, 48 are sufficiently close, the signals received by the controller 60 may provide an indication of the alignment such that mapping may proceed even with the active electrodes 48 within the sheath 10. In one example, the signals received by the controller 60 may be presented on a display (not shown) coupled to the controller 60, and the operator may visually monitor the signals to determine when the electrodes 20, 48 are sufficiently aligned. In addition or alternatively, the controller 60 may analyze the signals automatically to determine when the electrodes 20, 48 are sufficiently aligned and provide an output, e.g., on a display or other output device, to inform the operator.

In addition or alternatively, one or more of the active electrodes 46, 48 may be configured for delivering electrical energy to adjacent tissue, e.g., to perform an ablation on tissue contacted by or sufficiently close to the pass-through electrodes 20. For example, the controller 60 may include a source of electrical energy (not shown) configured to deliver energy to one or more of the active electrodes 46, 48. When the pass-through electrodes 20 are sufficiently aligned with the active electrodes 46, 48 and are positioned adjacent target tissue, the controller 60 may be activated to deliver energy to ablate target tissue, as described elsewhere herein.

In one example, the catheter 40 may be a bipolar ablation catheter, and the proximal-most electrode 48 may function as a return path for the current. This proximal-most electrode may be frequently pulled back into the sheath 10 in common ablation situations. In this application, a pass-through electrode on the sheath, e.g., the electrode closest to the distal tip 15, may be aligned with the retracted proximal-most return electrode to allow return current to pass through the pass-through electrode to the return electrode. Optionally, in this example, the sheath may include one or more additional pass-through electrodes, e.g., for ablation pass-through and/or for sensing pass-through. Using this configuration it may be possible to operate with less length of the ablation catheter extended from the sheath, e.g., shaped or deflectable, which may in turn provide for greater maneuverability, e.g., within a chamber of a patient's heart.

Alternatively, the catheter 40 may be a combination mapping and ablation device, e.g., with the ring electrodes 48 configured to provide mapping and with the tip electrode 46a and distal-most ring electrode, 46b configured to perform ablation. For example, the distal-most ring electrode 46b may provide a return electrode for bipolar ablation using the tip electrode 46a.

In another example, the sheath 10 may be a slittable or peelable sheath, such as those used for lead delivery, and include one or more pass-through electrodes 20. Similar to mapping or ablation catheters, many leads have electrodes on their distal portions for sensing, which become non-useful when retracted into an outer sheath. In this case, the normal condition is retracted, as the leads themselves are optimized for mechanical and electrical implantation, not for positioning. Further, many of these leads have fixed or retractable screws which prevent the desired navigation and movement of the sheath (while hunting for the ideal anatomical or electrical location for implantation) if they were even partially extending from the end of the delivery system. Thus, using the sheath 10 positioned around a lead, with the lead retracted just slightly into the distal end 14, the pass-through electrode(s) may be aligned with the lead electrode(s) at the corresponding spacing to the typical preferred position, and the lead may be used for sensing, mapping, etc., while remaining within the sheath 10.

Further, including traditional electrodes is even more challenging in a peelable/slittable sheath as all of the prior described difficulties apply as well as additional ones relating to maintaining the properties of the device to be peelable/slittable. In this case, ideally, each pass-through electrode may be formed as a point/spot electrode or a ring electrode of a soft metal capably of being slit, or in the case of a peelable sheath, at least partially pre-slit.

With reference again to FIGS. 1A and 1B, the quantity and position of the pass-through electrodes 20 may be optimized based on the expected procedure catheter or device that is to be introduced through the sheath 10. For example, the spacing between the pass-through electrodes 20 may substantially match the spacing between active electrodes 46 on the catheter 40. Optionally, the distal-most pass-through electrode 20 may be offset proximally from the outlet 17 of the sheath 10, e.g., corresponding to an expected offset of the active electrodes 46 of the procedure catheter or device 40 when the catheter 40 is retracted to a typical location as used clinically.

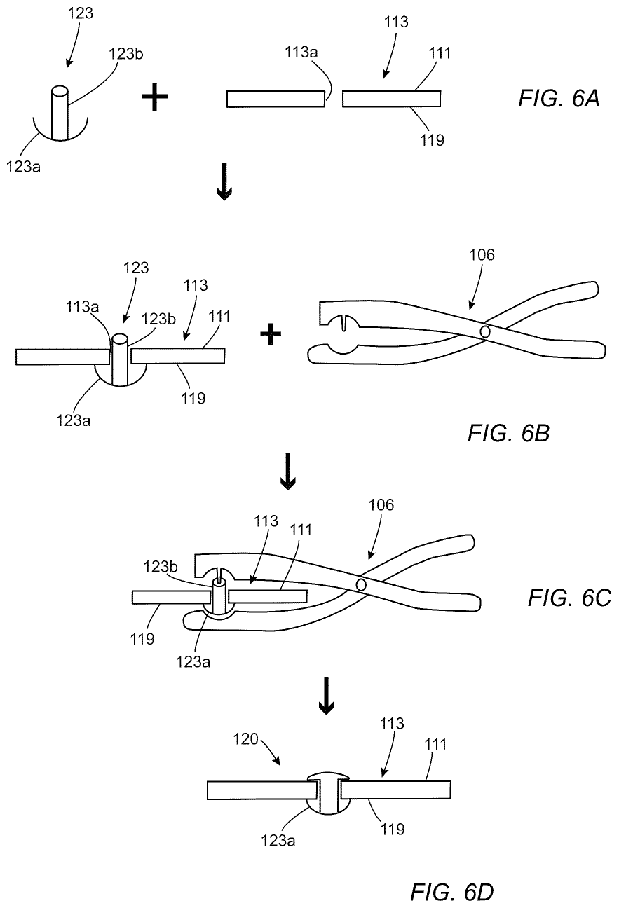

In the event of multiple ideal retracted positions of the procedure catheter 40 (or impedance electrodes on a dilator, e.g., as shown in FIGS. 6A and 6B, when fully inserted into the sheath 10), one or more additional pass-through electrodes may be provided on the sheath 10 at the same spacing so that the pass-through functionality is fully enabled in two or more retracted positions.

In another alternative, e.g., as shown in FIG. 5, a substantially continuous set of pass-through electrodes 320 may be provided on the distal end 314 of a sheath or other tubular member 310. The sheath 310 may include a plurality of pass-through electrodes 320 spaced apart at a distance shorter than the spacing of the active electrodes on the procedure device. For example, the pass-through electrodes 320 may be evenly spaced at a distance that is a fraction of the spacing of the active electrodes, e.g., one half, one third, one quarter, and the like, such that, at multiple positions, the active electrodes will be aligned with or proximate to a subset of the pass-through electrodes 320. For example, the spacing of the pass-through electrodes may ensure that, at any given retracted position of the procedure device, there are pass-through electrodes sufficiently close to each of the active electrodes, e.g., to provide effective position sensing of the procedure device relative to surrounding anatomy.

Optionally, in any of the sheaths or tubular devices herein including pass-through electrodes, the devices may also include one or more “active” electrodes, e.g., one or more “hard wired” traditional electrodes coupled to one or more conductors (not shown) extending from the distal end to the proximal end of the device. In one example, a sheath may include a plurality of impedance-based mapping electrodes, e.g., three electrodes, in a deflection area of the sheath that align with the mapping electrodes of common mapping and ablation catheters (such as the Biosense Smarttouch devices), and a fourth electrode near a distal tip of the sheath that is a combination hard-wired and pass-through electrode. This pass-through electrode may be configured for performing ablation, e.g., configured to be aligned with a proximal electrode of a bipolar set of ablation electrodes provided on a procedure device introduced through the sheath. Thus, the fourth electrode may be used to pass through ablation from the procedure device inside the sheath and may also be used as a fiducial mapping electrode to provide a mapping electrode not subject to precise alignment of the internal mapping/ablation catheter inside it with the pass through electrodes. In a variation of this construction, the combination electrode may instead be the distal-most electrode of three electrodes leaving the distal-most pass-through electrode dedicated to pass through ablation energy from an internally aligned active ablation electrode.

Although multiple hard-wired electrodes may be provided on a sheath or tubular device including pass-through electrodes, generally it may be desirable to include only a single combination electrode or single hard-wired electrode. This is because in electrophysiology suites or catheter labs, physicians, nurses, scrub techs, field engineers and the like are familiar with using alligator clips or other simple ways to connect into lab equipment. This means that a very simple pin or tab may be added to the handle of the sheath (which connects to the single hard wired electrode) in a convenient location that the operator may connect to. This eliminates an expensive connector and cable keeping the device costs and manufacturability in check.

Turning to FIG. 2A-2C, in one example, each pass-through electrode 20 may include a ring-shaped or annular outer portion 22 and a spot or point inner portion 24. Such a configuration may be useful for applications where it is desired to distribute electrical fields around the sheath 10, e.g., during an ablation procedure. For example, the spot or point inner portion 24 may provide sufficient surface area to electrically couple an active electrode axially aligned with the pass-through electrode 20 to the annular outer portion 22 (via the wire or conductor 26).

The outer portion 22 may be an enclosed ring that is secured around the outer wall 11 of the sheath 10 at a desired location on the distal end 14, e.g., by one or more of interference fit, bonding with adhesive, sonic welding, fusing, and the like. Optionally an annular groove or other recess may be provided in the outer wall 11 such that the ring 22 is at least partially recessed into the outer surface 11 and/or the sheath material may be softened or reflowed to seat the ring 22 within the outer wall 11, e.g., such that the ring 22 is flush with the outer surface 11. As best seen in FIGS. 2B and 2C, the inner portion 24 may be a rivet-like spot electrode (or array of electrodes) placed on the inner surface of the lumen 18a with a dome or other head defining the inner portion 24 and a body or shaft 26 extending through the wall of the sheath 10 to electrically couple the head/inner portion 24 to the ring/outer portion 22.

In one method, the rivet-like spot electrode 24 may be positioned first within the lumen 18a and the body or shaft 26 may be inserted through the wall until exposed from the outer wall 11. A ring electrode (or array of electrodes) may then be placed over or into the outer wall 11 such it contacts the body or shaft 26, and then the components may be fixed into place in a way that maintains electrical contact between the outer and inner portions 22, 24, e.g., one or more of swaging, crimping, gluing, fusing, and the like. The shaft 26 may be electrically insulated between the outer and inner portions 22, 24, i.e., to isolate the shaft 26 from wires or other components within the wall of the sheath 10, while electrically coupling the outer and inner portions 22, 24.

In another method shown in FIG. 2D, a shaft 26′ may be provided on an inner surface of a ring electrode portion 22′, which may be received through an opening in the wall of the sheath 10. A tip 24′ of the shaft 26′ may extend into the lumen 18 of the sheath 10 or may be substantially flush with the inner wall 19 to provide the inner portion of the pass-through electrode 20.′ The shaft 26′ may be attached to the ring electrode 22′ before being mounted to the sheath 10 or, alternatively, may be formed separately and inserted through the opening into contact with the ring electrode 22,′ and permanently attached to the sheath 10 and/or ring electrode 22′ to provide the pass-through electrode 20.′

In another method, one or more wire-like insulated conductors may be placed through the wall of the tubular member to provide the conductor 26 extending through the wall of the sheath 10, and an inner end of the conductor(s) may be bent over on the inner surface of the lumen 18a and fixed to the wall, e.g., by one or more of bonding, fusing, and the like. The inner face of the conductor(s) may then be abraded or otherwise treated to expose the conductive material of the conductor(s) and, thus create an inner portion or spot electrode. Optionally, a larger surface spot electrode may be attached to the inner surface and coupled to the inner end of the conductor(s), if desired to provide additional surface area for the inner portion 24.

One or more ring electrodes may then be placed over the passage point of the conductor(s) and fixed into place both mechanically and electrically to the conductor(s) to provide the outer portion 22. Alternatively, the conductor(s) may be wrapped one or more times around the outer surface 11 of the sheath 10, e.g., within an annular groove (not shown) formed in the outer wall to form a ring. The outer insulative surface of the resulting loop(s) may be abraded or otherwise treated to complete the pass-through electrode 20. Optionally, additional conductive material may be applied over the loop(s) if desired to enhance conductivity of the outer portion.

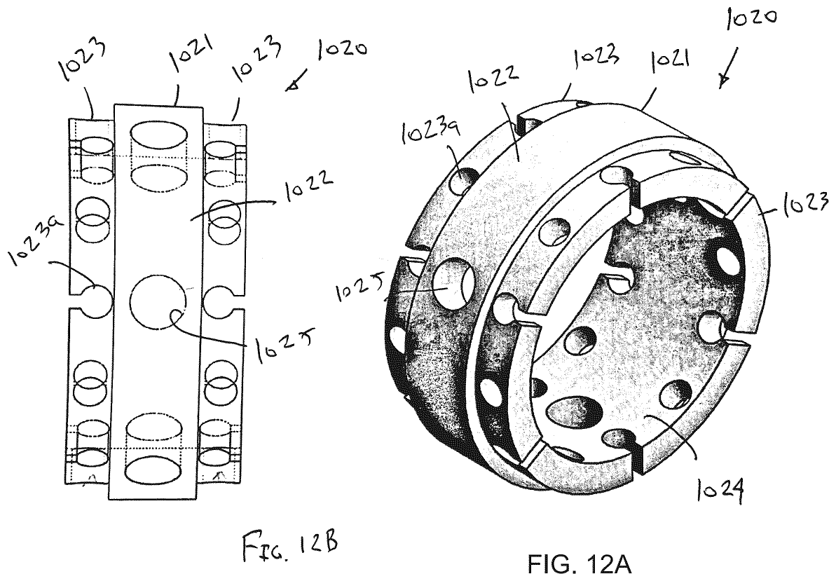

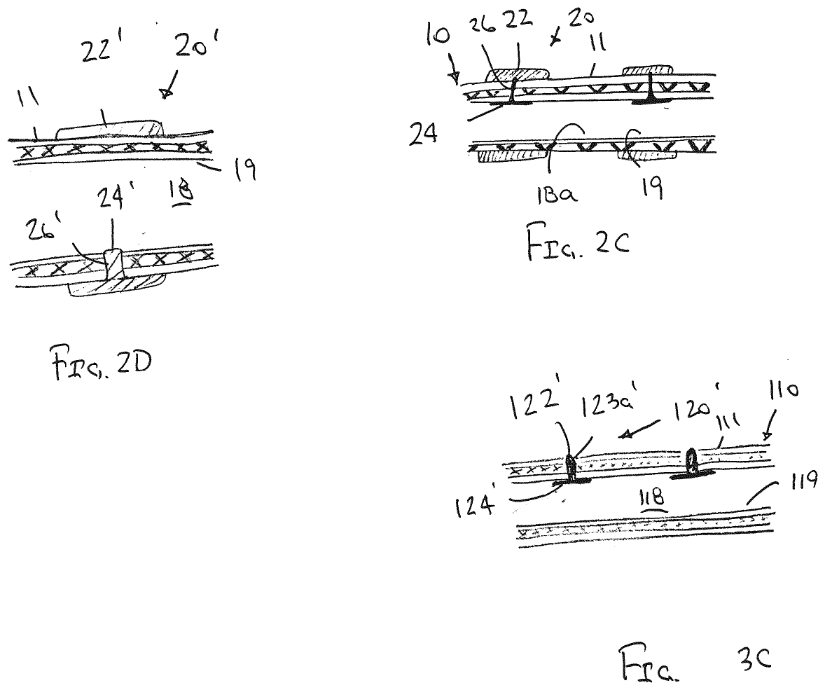

In yet another example, the sheath may include one or more pass-through electrodes formed as a ring that has the thickness substantially the same as the wall thickness of the distal end 14 of the sheath, e.g., as shown in FIGS. 12A and 12B. In one method, one or more extrusions defining the distal end may be reflowed into the proximal and distal ends of each pass-through electrode, e.g., to attached the electrode and/or electrically insulate the electrode from components within the wall, e.g., braided elements of a reinforcing layer and/or pull wire(s). In one example, one or more mechanical holes or grooves (not shown) may be provided in the ring electrode, leaving the center of the electrode electrically conductive from the outer portion 22 to the inner portion 24. Optionally, one or more irrigation holes may be provided in the center of the electrode or otherwise communicating between the outer and inner portions 22, 24 to allow fluid to pass through the electrode 20 from the lumen 18a.

Alternatively, a ring electrode used for the pass-through electrode may be electrically connected to one or more conductors within the distal end. For example, conductors may be coupled to the electrode and pass axially along the wall of the sheath, e.g., either triaxially braided into reinforcement members in the wall and/or non-triaxially braided between the lumen liner and the extrusions. In a further alternative, one or more of the semi-continuous pass-through electrodes may be provided that are grounded to a common central conductor (such a wire or other member of a reinforcement layer of the sheath. In a further alternative, the outer surface of the sheath 10 may be made electrically conductive over at least a portion of its length, which may enable impedance-based mapping of a procedure device within the sheath, when positioned in the blood pool within a patient's body.

Turning to FIGS. 3A and 3B, in another example, a sheath 110 is provided that includes a plurality of spot or point pass-through electrodes 120. In this example, both the outer portion 122 and the inner portion 124 may be formed as relatively small conductive regions, e.g., having a circular, elliptical, square, or other cross-sectional surface area, against the outer and inner surfaces 111, 119 of the sheath 110. As shown in FIG. 3A, the pass-through electrodes 120 may be aligned axially with one another i.e., in a linear array substantially parallel to the longitudinal axis 116. Optionally, multiple linear arrays may be provided that are spaced apart from one another around the circumference of the distal end 114 of the sheath 116 (not shown). In this option, each array may include electrodes at the same axial position such that the arrays also define circumferential arrays. Alternatively, one or more of the arrays may be offset axially from one another such that electrodes of at least some linear arrays are offset axially from other linear arrays.

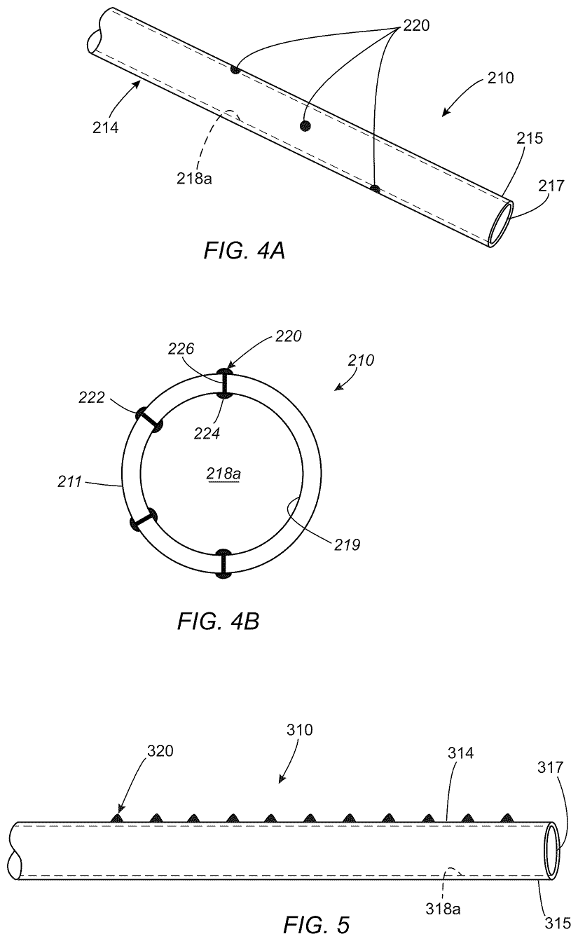

In another alternative, shown in FIGS. 4A and 4B, a sheath 210 may be provided that includes a plurality of pass-through electrodes 220 that are offset axially and circumferentially from one another. For example, as shown in FIG. 4A, the electrodes 220 may be arranged in a spiral array along a length of the distal end 214.

Turning to FIGS. 6A-6D, an exemplary method is shown for forming a pass-through electrode 120 that includes spot or point electrodes for both the outer portion 122 and the inner portion 124, such as that shown in FIGS. 3A and 3B. Initially, a wall 113 of a distal end 114 of a sheath or other tubular member 110 is provided. The wall 113 may be part of an entire sheath 110 that has already been fabricated or may be a relatively short extrusion or other tubular body that may be formed separately and attached to another longer tubular body to provide the distal end 114.

As shown in FIG. 6A, a hole or other opening 113a may be formed through the wall 113, e.g., by one or more of punching, drilling, cutting, and the like. Alternatively, if the wall 113 is part of an extrusion or other separate tubular body, the opening 113a may be formed when the tubular body is formed, e.g., by molding, casting, 3D printing, and the like. A rivet-like member 123 may be provided that includes a round otherwise-shaped head 123a corresponding to the inner portion 124 and a tubular shaft 123b extending from the head 123a and corresponding to the conductor 126.

Turning to FIG. 6B, the shaft 123b of the rivet-like member 123 may be inserted through the opening 113a from the inside the lumen 118 to place the head 123a against the inner wall 119, e.g., such that the shaft 123b extends about the outer surface 111 of the sheath 110. Then, as shown in FIG. 6C, a riveting dye or other tool 106 may be used to fold the exposed shaft 123b and/or otherwise deform the shaft 123b to provide the outer portion 122 of the electrode 120. The tool 106 may be further actuated as desired to compress the outer and inner portions 122, 124 against the outer and inner surfaces 111, 119 to provide the finished pass-through electrode 120, e.g., as shown in FIG. 6D.

Alternatively, as shown in FIG. 3C, a rivet-like member 123′ may be provided that includes a head 124′ and a shaft 123a′ that extends from the head 124.′ With the rivet-like member 123′ within the lumen 118 of the sheath 110, the shaft 123a′ may be inserted through an opening created in the wall of the sheath 110 such that a tip 122′ of the shaft 123a′ is exposed from the outer surface 111 of the sheath 110. In this alternative, the shaft 123a′ may have a rounded or flat tip 122′ shaped to extend from or remain flush with the outer surface 111 to provide the outer portion of the electrode 120.′ The head 124′ may be permanently attached to the inner surface 119 of the sheath 110, e.g., by one or more of bonding with adhesive, sonic welding, reflowing the sheath material, and the like.

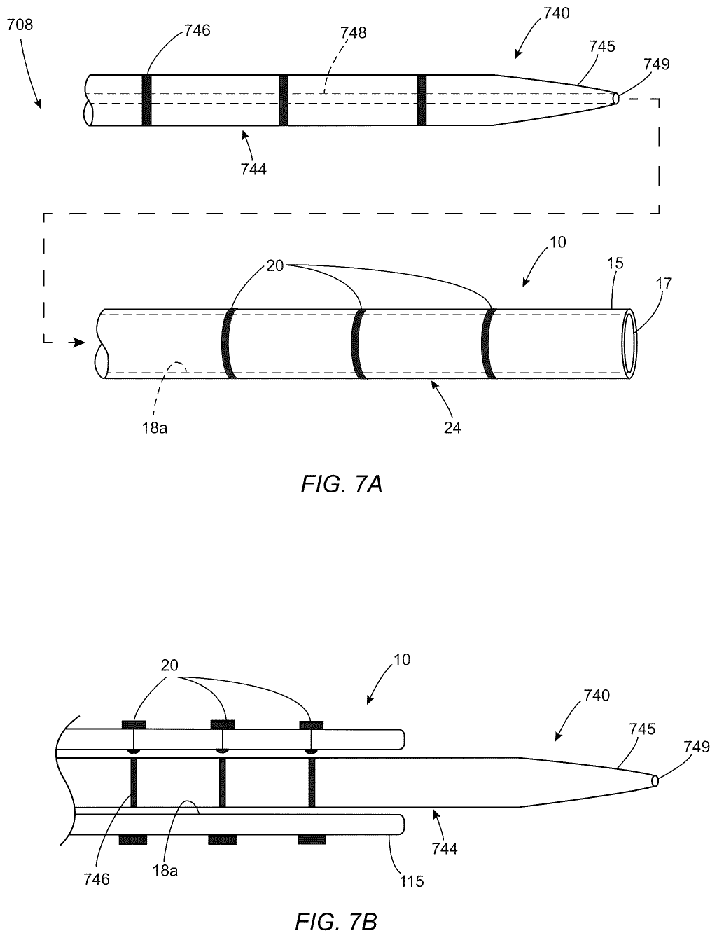

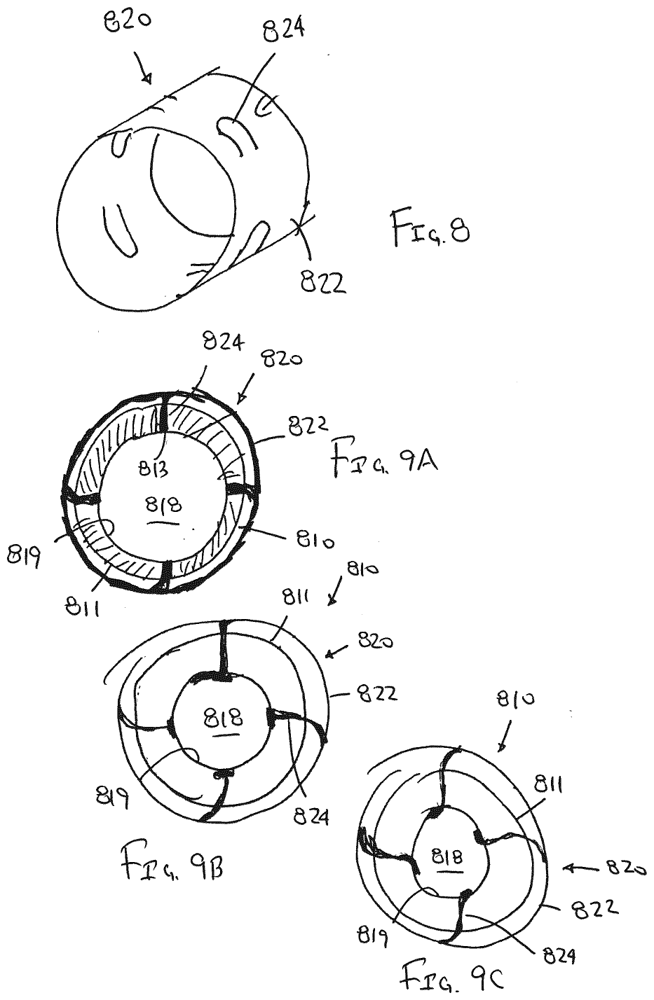

Turning to FIGS. 7A and 7B, another example of a system 708 is shown for performing a medical procedure that includes an outer sheath or other tubular member 10, which may be similar to the sheath 10 shown in FIGS. 1A and 1B or, alternatively, any of the other sheaths or tubular members herein, i.e., including one or more pass-through electrodes 20. In addition, the system 708 includes a dilator 740 including a proximal end (not shown), a distal end or portion 744 sized for introduction into a lumen 18 of the sheath 10, and a plurality of active electrodes 746 spaced apart from one another. As shown, the electrodes 746 may be spaced apart proximally from a tapered distal tip 745 of the dilator 740, e.g., sized and/or shaped to facilitate advancing the dilator 740 through an opening in tissue (not shown), e.g., to dilate the opening and/or otherwise facilitate introducing one or more instruments or devices through the tissue. Optionally, the dilator 740 may include a lumen 748 extending from its proximal end to an opening 749 in the distal tip 745, e.g., to slidably receive a guidewire or other rail. The size and shape of the distal tip 15 of the sheath 10 may provide a substantially smooth transition to the tapered distal tip 745 of the dilator 740, e.g., such that the entire system 708 may be advanced together through an opening in tissue, with the distal tip 745 dilating the tissue to accommodate the sheath 10.

In one example, the active electrodes 746 may be used for mapping or other sensing. When the dilator 746 is positioned within the sheath 10 with the active electrodes 746 aligned with the pass-through electrodes 20, the dilator 746 may be used for mapping or other sensing as if the sheath 10 were not present given the pass-through electrodes 20, similar to other devices and systems herein.

Alternatively, other devices may be provided instead of the dilator 740 that include one or more active electrodes, which may be introduced into the sheath 10 and deployed from the distal end 14 to perform a procedure and/or withdrawn into the sheath 10 to align active electrodes on the device with the pass-through electrodes 20. For example, a central line or pic line catheter may be provided that includes one or mapping or sensing electrodes (not shown) that may be introduced through the lumen 18a of the sheath 10. The electrodes on the catheter may be deployed from the distal end 14 during a procedure or positioned within the distal end 14 to align electrodes on the catheter with the pass-through electrodes 20.

In other alternatives, it may be desirable to make construction of the pass-through electrodes as simple as possible. For example, it may be desirable to make the construction of each pass-through electrode as a single piece. Among the single piece construction variations, of particular value is a “tongue” or “foot” or “leg” cut out of a classic ring electrode.

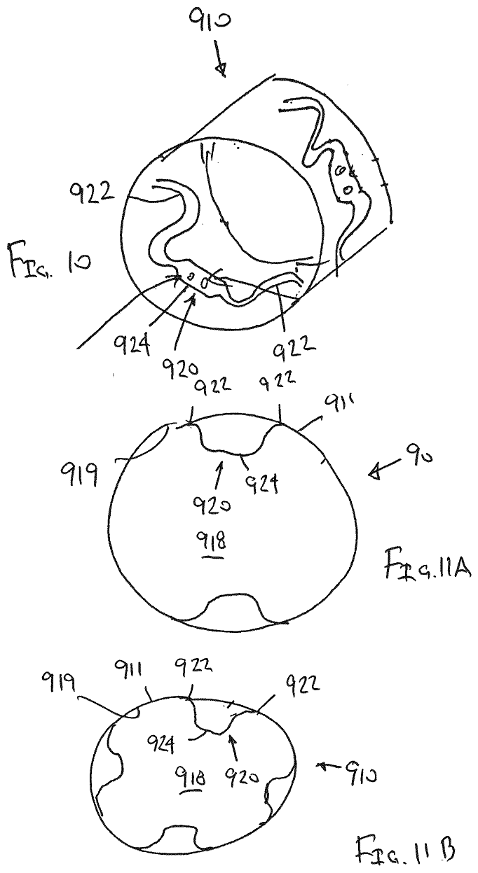

For example, FIG. 8 shows an example of a single piece electrode 820 that may be formed and mounted to a sheath or other tubular member, such as the sheath 810 shown in FIGS. 9A-9C. As shown, the electrode 820 includes an annular sleeve 822 including one or more tabs 824 formed in the wall. In one method, the sleeve 822 may be formed from a flat sheet with the tabs 824 formed therein, e.g., by one or more of stamping, laser cutting, etching, and the like, and then the sheet may be rolled and the edges of the sheet attached together, e.g., by welding, fusing, bonding with adhesive, and the like, to provide the sleeve 822. Alternatively, the electrode 820 may be formed as a tubular body, e.g., by one or more of extruding, molding, casting, and the like, and the tabs 824 may be formed in the tubular body, e.g., by one or more of laser cutting, etching, and the like.

As shown in FIG. 8, the electrode 820 includes four tabs 824 spaced apart from one another, e.g., substantially uniformly around circumference of the sleeve 822. Alternatively, the sleeve 822 may include other arrangements of tabs, e.g., including one or more tabs spaced evenly or around the circumference and/or with multiple tabs positioned adjacent one another between the opposite edges of the sleeve 822 at the same circumferential location.

To incorporate the electrode 820 into a sheath or other tubular member 810, one or more slots or other openings 813 may be created through the sheath 810, e.g., spaced apart around the circumference of the sheath wall at a spacing corresponding to the spacing of the tabs 824. The sleeve 822 may be positioned around the sheath 10 and the tabs 824 may be bent or otherwise directed through the openings 813, such that the tabs 824 extend into the lumen 818 of the sheath 810. Alternatively, if the electrode 820 is formed from a flat sheet, the flat sheet may be wrapped around the sheath 810 and the tabs 824 inserted into the openings 813. The opposite ends of the flat sheet may then be attached together or simply positioned adjacent one another on the outer wall of the sheath 810. The electrode 820 may be permanently attached to the sheath 810, e.g., by one or more of an interference fit, bonding with adhesive, reflowing the sheath material, fusing, sonic welding, and the like.

The tabs 824 may have sufficient length to extend through the wall of sheath 810 such that tips of the tabs 824 provide the inner portion of the pass-through electrode 820. For example, as shown in FIG. 9A, the tabs 824 may have a length corresponding to the thickness of the wall of the sheath 810 to provide an inner portion of the electrode 820 that is flush with the inner surface 819 of the sheath 810. Alternatively, as shown in FIGS. 9B and 9C, the tabs 824 may extend into the lumen 818 and then deformed against the inner wall 819. For example, in FIG. 9B, the tips of the tabs 824 may be flattened or mushroomed against or within the inner wall 819, while in FIG. 9C, the tips of the tabs 824 are bent over against the inner wall 819. Optionally, surfaces of the tabs 824 may be electrically insulated at least partially between the sleeve 822 and tips to isolate the tabs 824 from wires or other components within the wall of the sheath 810.

In another example, the proximal of the two main bipolar ablation electrodes is aligned with the most distal pass-through electrode, with all the additional electrodes then offset based on that position and alignment with the remaining electrodes of the procedure catheters, and then the electrode with tongue cut-out(s) may be aligned over respective openings through the sheath wall and the tongue(s) may be depressed into the opening(s) (in combination with rotation as needed) until the tongue (foot/leg/etc.) hits a mandrel (not shown) positioned within the lumen of the sheath. In another example, the foot (or alternatively the hole or opening through the sheath wall) is electrically insulation or otherwise isolated from the braid through which the hole goes. The electrode assembly is then fixed into place by one of many common methods including 1) swaging, 2) gluing (hole and edge glue) and the process repeated for the other electrodes.

Alternatively, the tongue/foot may instead be cut out more like a stent structure with a total cut pathlength much more than the radial arc length such that it can be depressed/extended down into the hole without rotation or the like.

Alternatively, each electrode may include multiple tongues/feet/legs (to go in multiple holes) to either increase the stability of the electrode and/or the surface area of inner contact and/or uniformity/integrity of signal or ablation transmission.

In yet another alternative, the pass-through electrode(s) may be made with a “tack” like element. The base of the “tack” is displaced against the inner wall of the sheath with the “stem” of the tack passing through the shaft. The stem itself (at its exit point of the shaft) may be the effective electrode, or alternatively, a ring electrode may be placed (e.g., swaged, glued, etc.) around the exit location while in electrical contact with the ring.

For example, turning to FIG. 10, exemplary electrodes 920 are shown that include a central tack region 924 and a pair of tabs 922 extending from opposite sides of the tack region 924. The tabs 922 may be bent or otherwise shaped, and then inserted through respective holes or openings through the wall of the sheath or tubular member 910, e.g., until exposed on the outer wall 911 of the sheath 910. The tabs 924 and/or tack region 924 may be attached to the sheath 910, e.g., by one or more of interference fit, bonding with adhesive, fusing, and the like to permanently attach the electrode 920 to the sheath 910. In the example shown in FIG. 11A, two electrodes 920 are provided on the sheath 910 with the tack region 924 spaced away from the inner wall 919 of the sheath 910 and the tabs 922 extending through the wall, while in FIG. 11B, four electrodes 920 are provided.

The outer ends of the tabs 922 may extend from the outer wall and/or be pressed or otherwise positioned flush on the outer wall. The exposed outer ends may be sufficient to provide the outer portion of the electrode 920 or, alternatively, an additional electrode member or conductive material may be applied to the outer surface 911 that is electrically coupled to the ends of the tabs 922. For example, an annular member (not shown) may be attached around the outer surface 911 and coupled to the tabs 922 or one or more spot or point electrodes may be attached to the outer surface 911, e.g., with a single spot electrode coupled to each pair of tabs or separate spot electrode coupled to each of the tabs (also not shown).

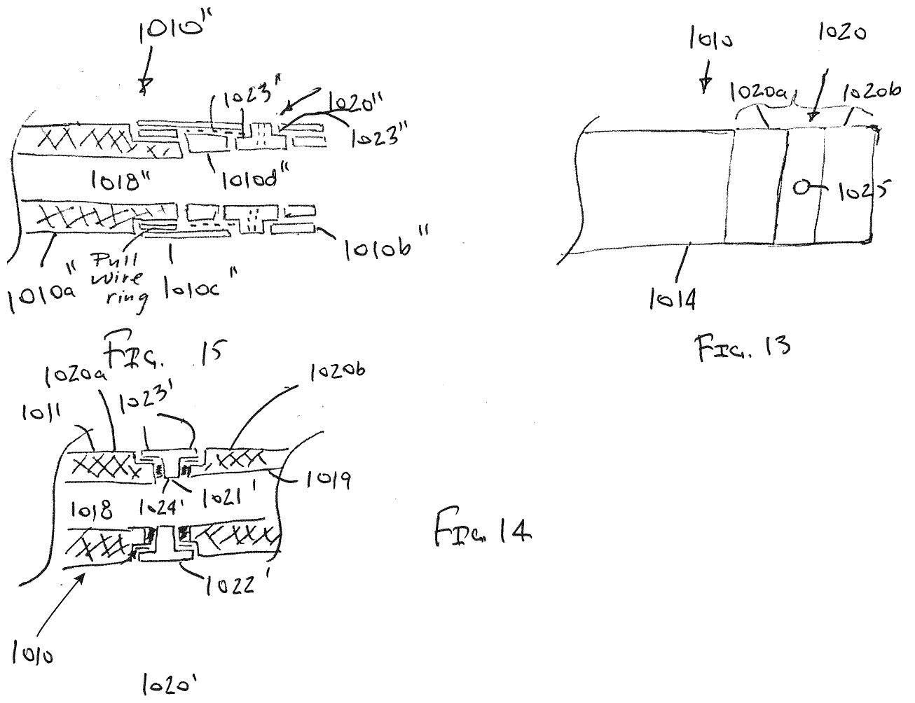

Turning to FIGS. 12A and 12B, another example of a ring electrode 1020 is shown that may be attached to a sheath or other tubular member, e.g., the sheath 1010 shown in FIG. 13. At least a portion of the electrode 1020 may have a thickness substantially the same as the wall thickness of the distal end 1014 of the sheath 1010. For example, as shown in FIGS. 12A and 12B, the electrode 1020 may include a central annular region 1021 including an outer surface 1022 that may be exposed or flush with the outer surface of the sheath and an inner surface 1024 that may be exposed or flush with the inner surface of the sheath when the electrode 1020 is mounted or otherwise attached to the sheath.

In the example shown, the electrode 1020 includes collars 1023 on opposite sides of the central region 1021 that have a smaller thickness than the central region 1021, e.g., such that the sheath may be attached to the collars 1023, e.g., received over the collars 1023 and then reflowed or otherwise attached to the collars 1023. For example, as shown, each collar 1023 may include one or more holes, recesses, or other features 1023a into which material from the adjacent sheath sections 1020a, 1020b may flow or otherwise engage the collars 1023 to permanently attach the collars 1023 to the adjacent sheath sections, e.g., with the outer and inner surfaces 1022, 1024 flush with the outer/inner walls of the sheath. Optionally, the central region 1021 may include one or more holes or other passages 1025 therethrough, i.e., extending between the outer and inner surfaces 1022, 1024. The passages 1025 allow fluid to flow therethrough, e.g., fluid introduced through the lumen of the sheath to flow out through the passages 1025, which may be used to cool the electrode 1020 and/or enhance electrically coupling the electrode 1020 to adjacent tissue.

Alternatively, as shown in FIG. 14, the ring-electrode 1020′ may include collars 1023′ on opposite sides of a central region 1021′ that are flush with the outer surface 1022.′ The collars 1023′ have a thickness smaller than the central region 1021, e.g., such that the electrode 1020′ may be attached to the adjacent sections 1010a, 1010b of the sheath 1010 by lap attachments, which may be reflowed or otherwise attached to the collars 1023′ similar to the previous examples.

In a further alternative shown in FIG. 15, a ring electrode 1020″ may be provided, e.g., similar to the electrode 1020 shown in FIGS. 12A and 12B, and the sheath 1010″ may include multiple extrusions or other sections 1020a″-1020d″ that may be attached to the collars 1023″ to attach the electrode 1020″ to the sheath 1010.″ Optionally, the multiple sections 1010a″-1010d″ may allow a conductor, e.g., a wire and the like, to be coupled to the electrode 1020,″ e.g., to provide a combination passive/active electrode, if desired, as described elsewhere herein.

The foregoing disclosure of various examples has been presented for purposes of illustration and description. It is not intended to be exhaustive or to limit the invention to the precise forms disclosed. Many variations and modifications of the embodiments described herein will be apparent to one of ordinary skill in the art in light of the above disclosure.

Further, in describing representative embodiments, the specification may have presented the method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the scope of the appended claims.

1. An apparatus for performing a procedure within a patient's body, comprising:
a tubular member comprising a proximal end, a distal end sized for introduction into a patient's body, a longitudinal axis extending between the proximal end and the distal end;
a primary lumen extending between the proximal end and the distal end; and
one or more pass-through electrodes on the distal end, each pass-through electrode including an outer portion exposed on an outer wall of the tubular member and an inner portion exposed within an inner wall of the primary lumen.

2. The apparatus of claim 1, wherein each pass-through electrode is passive.

3. The apparatus of claim 2, wherein the only conductive pathway for each pass-through electrode is between the outer portion and the inner portion.

4. The apparatus of claim 2, wherein each pass-through electrode is electrically isolated from other electrodes or components on the tubular member.

5. The apparatus of claim 2, wherein each pass-through electrode is unconnected to any wires or conductors on the tubular member.

6. The apparatus of claim 1, wherein each pass-through electrode comprises a wire or conductor extending through the sheath between the outer portion and the inner portion.

7. The apparatus of claim 6, wherein the wire or conductor is electrically isolated from surrounding material of the sheath.

8. The apparatus of claim 6, wherein the wire or conductor extends radially through the sheath between the outer portion and the inner portion.

9. The apparatus of claim 1, wherein the one or more pass-through electrodes comprise a plurality of pass-through electrodes spaced apart from one another on the distal end.

10. The apparatus of claim 9, wherein the pass-through electrodes are spaced apart axially from one another.

11. The apparatus of claim 10, wherein the pass-through electrodes comprise a plurality of spot electrodes aligned axially with one another.

12. The apparatus of claim 10, wherein the pass-through electrodes comprise a plurality of spot electrodes spaced apart circumferentially from one another.

13. The apparatus of claim 12, wherein the spot electrodes are arranged in a spiral configuration on the distal end.

14. The apparatus of claim 1, wherein each pass-through electrode includes an annular outer portion extending circumferentially around the outer wall of the sheath.

15. The apparatus of claim 14, wherein each pass-through electrode includes an annular inner portion extending circumferentially around the inner wall.

16. The apparatus of claim 15, wherein each pass-through electrode comprises a ring electrode.

17. The apparatus of claim 16, wherein the ring electrode has a radial thickness substantially the same as a radial thickness of the tubular member between the outer wall and the inner wall.

18. The apparatus of claim 14, wherein each pass-through electrode includes a spot electrode defining the inner portion.

18-32. (canceled)

33. A dilator for providing access into a body lumen of a patient, comprising:
a tubular member comprising a proximal end, a distal end sized for introduction into a patient's body and terminating in a tapered distal tip for advancing the distal end through an opening in tissue, a lumen extending between the proximal and distal ends; and
one or more active electrodes on the distal end configured to perform a diagnostic or therapeutic procedure.

34-40. (canceled)

41. A system for performing a procedure within a patient's body, comprising:
a tubular member comprising a proximal end, a distal end sized for introduction into a patient's body to a target location, a lumen extending between the proximal end and the distal end, and one or more pass-through electrodes on the distal end, each pass-through electrode including an outer portion exposed on an outer wall of the tubular member and an inner portion exposed within an inner wall of the primary lumen; and
a procedure device comprising a distal portion introducible through the lumen to deploy one or more active electrodes on the distal portion within the target location to perform a diagnostic or therapeutic procedure, the one or more electrodes configured to be aligned with the one or more pass-through electrodes when the distal portion is positioned at least partially within the distal end of the tubular member.

42-70. (canceled)

71. A method for making a pass-through electrode on a tubular member, comprising:
providing a tubular body including a wall surrounding a lumen;
positioning a rivet-like member including a conductive head and a shaft extending from the head within the lumen;
inserting the shaft through an opening extending through the wall until the shaft is exposed at an outer surface of the wall and the head is positioned against an inner surface of the wall; and
forming an outer portion on the outer surface that is electrically coupled to the head.

72-86. (canceled)

87. A method for performing a medical procedure, comprising:
introducing a distal end of a tubular member into a body lumen within a patient's body, the distal end including one or more pass-through electrodes providing a conductive pathway between an outer surface of the distal end and an inner surface of a lumen within the tubular member;
introducing a distal end of a procedure device into the lumen such that one or more active electrodes on the procedure device distal end are positioned within the tubular member distal end;
positioning the procedure device such that the one or more active electrodes are aligned with or proximate to respective one or more pass-through electrodes on the tubular member; and
delivering electrical signals to the one or more active electrodes, the pass-through electrodes transmitting the signals to tissue surrounding the tubular member distal end.

88-97. (canceled)
